(19) United States
(12) Patent Application Publication
(10) Pub. No.: US 2020/0114012 A1
(43) Pub. Date: Apr. 16, 2020

(54) METHOD FOR PRODUCING A LIQUID COMPOSITION

(71) Applicants: The University of Liverpool, Liverpool (GB); The Johns Hopkins University, Baltimore, MD (US)

(72) Inventors: Steven Paul Rannard, Liverpool (GB); Andrew Owen, Liverpool (GB); Paul Curley, Liverpool (GB); James Hobson, Liverpool (GB); Marco Siccardi, Liverpool (GB); Caren L. Freel Meyers, Baltimore, MD (US); Amer Al-Khouja, Baltimore, MD (US); David J. Meyers, Baltimore, MD (US); Charles Williams Flexner, Baltimore, MD (US)

(21) Appl. No.: 16/499,639

(22) PCT Filed: Mar. 29, 2018
(86) PCT No.: PCT/GB2018/050887

§ 371 Date: Sep. 30, 2019

(30) Foreign Application Priority Data
Mar. 30, 2017 (GB) 1705087.3

(51) Int. Cl. A61K 47/44; A61K 31/675; A61K 47/14; A61P 31/18

(52) U.S. Cl. CPC A61K 47/44; A61K 31/675; A61K 9/0019; A61P 31/18; A61K 47/14

(57) ABSTRACT

A first aspect of the present invention provides a method of producing a liquid composition comprising stabilised particles of active species in oil by precipitating a solution of the active species in a non-solvent in the presence of one or more stabilisers, mixing the precipitate suspension with an oil and then removing the solvents. Further aspects of the present invention relate to liquid compositions produced by said method and methods of use of such liquid compositions.

The present invention relates to a method of producing a liquid composition comprising stabilised particles of active species in oil, to a liquid composition produced by said method, and to a method of use of such liquid compositions.

### BACKGROUND OF THE INVENTION

There are many instances where the slow release of a pharmacologically active compound would be desirable. For example, many drugs, such as those taken for chronic conditions or as prophylactics, need to be taken on a regular schedule to maintain a therapeutically effective concentration and poor patient compliance reduces their efficacy. If such drugs could be administered in such a way as to maintain a therapeutically effective concentration without requiring action on the part of the patient, then the issue of poor patient compliance would be ameliorated.

One solution is to formulate the drug in such a way that the formulation releases the drug gradually, maintaining an efficacious concentration over a predetermined period of time. A particular example is to encase the drug in a polymer sheath, which slowly releases the drug. This approach is commonly used in contraceptive implants, for example as in U.S. Pat. No. 4,957,119 A.

However, this approach is not without its drawbacks. Using contraceptive implants as an example, medically trained professionals are required to insert and remove the polymer sheath, an unpleasant procedure which reduces uptake of the method among the eligible patient population.

Additionally, the pharmacokinetic properties of many drugs are enhanced when administered with high fat meals. This results in, for example, improved oral bioavailability of the drug. An improvement in oral bioavailability of the drug could result in a reduced oral dose still having the desired therapeutic effect, this reduction in oral dose could result in a corresponding reduction to adverse side effects and long-term toxicity.

Furthermore, patient compliance is also an issue for oral dosing regimens as patients must coordinate their meals, both in terms of timing and content, with their dosing regimen to gain this enhanced oral bioavailability. If the oil could be provided with the drug, then the enhanced bioavailability could be acquired without further inconvenience to the patient.

Lipid-soluble drugs may be dissolved in a lipid to enhance their oral bioavailability. Water-soluble (i.e. lipid-insoluble) drugs cannot be dissolved into lipids and hence cannot benefit from this enhancement to oral bioavailability. One possible solution would be to create solid drug nanoparticles (SDNs) of water-soluble drugs and disperse them in a lipid phase. However, SDNs of water soluble drugs are difficult to produce in an efficient manner, preventing this approach from being pursued effectively.

It was previously discovered (as described and claimed in WO2006/079410A1) that carrier liquids could be prepared by utilising a method comprising preparing an emulsion from a) an aqueous phase, b) a second liquid phase, which is volatile and immiscible with the aqueous phase, c) a carrier material, which is soluble in the continuous phase of the emulsion and liquid at ambient temperature, and d) a dopant material, which is soluble in the disperse phase of the emulsion, and subsequently cooling the emulsion until both the continuous phase and the carrier material become solid (i.e. they freeze), followed by removal of water and the volatile second phase from the cooled emulsion in vapour form and thawing to obtain a liquid product (at ambient temperature) with the dopant material dispersed therein.

This idea was subsequently developed to embrace an alternative method (as described and claimed in WO2013/030535A2) which did not utilise an emulsion. The alternative method comprises preparing a single phase solution comprising a) a solvent or mixture of miscible solvents, b) a liquid carrier soluble in solvent (a), and c) a dopant material which is also soluble in solvent (a); cooling the single phase solution such that the solvent (a) and liquid carrier (b) freeze; removing the solidified solvent in vapour form; and thawing to obtain a liquid product (at ambient temperature) with the dopant material dispersed therein.

The liquid products obtained from the above processes are suitable for the provision of a dopant material to a system comprising a solvent in which the dopant material is either insoluble or sparingly soluble in an easily dispersible form. As a result, the concentration of the dopant material in the system rapidly increases over a short period of time (of the order of seconds up to a few minutes) as the dopant material is quickly dispersed. However, due to the method of preparing such liquid products, certain combinations of materials may prove to be unsuitable (e.g. in terms of their solubility characteristics) for the above methodologies. Additionally, such methods produce compositions which are suitable for the fast release of dopant materials into solvents which they are poorly soluble in.

Drugs are compounds which are pharmacologically active (active species). As discussed by Jordheim, L. P.; Durantel, D.; Zoulim, F.; and Dumontet, C. in Nat. Rev. Drug Discov. 2013, 12, 447, active species include analogues of nucleosides and nucleotides, which are of interest for a variety of pharmacological applications. For example, nucleotide analogue reverse-transcriptase inhibitors (NtRTIs) and nucleoside analogue reverse-transcriptase inhibitors (NRTIs) are valuable classes of drugs used in the treatment of viral infections. They act to block the synthesis of double-stranded viral DNA from viral RNA by inhibition of reverse-transcriptase. Analogues of nucleosides and nucleotides are also under investigation for activity against, and as treatments for, cancers. They act to inhibit cancer cell growth and replication. For example, cytarabine (an analogue of cytidine) is used in the treatment of leukaemias and lymphomas. Analogues of nucleosides and nucleotides often encounter issues with resistance, poor oral bioavailability and long-term toxicity.

Examples of NtRTIs include tenofovir prodrugs which are nucleotide analogues, specifically analogues of adenosine monophosphate. NtRTIs are used in the treatment of chronic viral infections, most commonly HIV, as well as prophylactically in high risk individuals. Unusually for drugs of this class, tenofovir prodrugs have significant water solubility. Typically for drugs of this class, tenofovir prodrugs require a highly regimented dosing regimen to maintain efficacy. If the regimen is not maintained the disease can progress further in the patient, and increases the possibility of transmission.

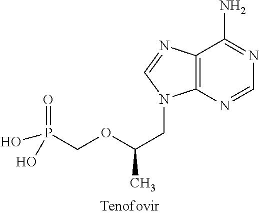 [SMILES: C[C@H](Cn1cnc2c(N)ncnc21)OCP(=O)(O)O]

An object of the present invention is to provide a method of producing a liquid composition, said composition comprising stabilised particles of at least one active species in an oil, as well as provision of such a liquid composition per se, where the at least one active species is released gradually, along with applications thereof, all of which obviate or least mitigate some of the problems or difficulties encountered in the prior art. A further object is to provide a liquid composition comprising stabilised particles of at least one active species, where the oral bioavailability of the active species is enhanced.

### SUMMARY OF THE INVENTION

### First Aspect

A first aspect of the present invention provides a method of producing a liquid composition comprising stabilised particulates of at least one active species in an oil, the active species being selected from nucleoside analogues and nucleotide analogues the method comprising the steps of:
1) dissolving the at least one active species into a first solvent to form a first solution;
2) dissolving one or more stabilisers into a second solvent to form a second solution, wherein the first and second solvents are miscible and the at least one active species is insoluble in the second solvent;
3) combining the first and second solutions to form a particulate suspension of stabilised particulates of the at least one active species suspended in the mixed solvent;
4) adding an oil to the particulate suspension to form a liquid mixture, wherein the at least one active species is insoluble in the oil; and
5) removing the first and second solvents from the particulate suspension to form the liquid composition.

The first aspect of the present invention is a method directed towards the production of a liquid composition. The liquid composition is comprised of an oil, in which are suspended stabilised particulates of at least one active species which are oil-insoluble.

For the avoidance of any doubt, the at least one active species could be one or more nucleoside analogue, one or more nucleotide analogue, or one or more of each of a nucleoside analogue and a nucleotide analogue.

By prodrug, it is meant a medication or compound that undergoes a biotransformation (e.g. metabolism) before exhibiting its pharmacological effects. Prodrugs often exhibit improved adsorption, distribution, metabolism and excretion characteristics compared to the parent drug as well as improved bioavailability.

For the avoidance of any doubt, throughout this specification by “liquid” and like terms it is meant the state of matter in which the substance in question exhibits (at a temperature above its solidification temperature but at or below 40° C.) a characteristic readiness to flow and relatively high incompressibility; the substance in question does not resist change of shape but does resist a change of size. Thus gels, waxes and other such “semi-solid” materials are to be considered (by virtue of the definition provided above) as substances which are “liquids” for the purposes of the present invention.

By “oil-insoluble” it is meant that the active species is not normally soluble in the oil, i.e. a solid body of the active species introduced into the liquid (in an amount of 1 mg/mL) will remain as such (i.e. solid) without dissolving.

By stabilised it is meant that the tendency of the particles to agglomerate while suspended in a liquid has been ameliorated or relieved entirely. As a consequence, the stabilised particulates do not flocculate and maintain their independent character and motility. Without wishing to be bound by theory, it is thought that on mixing the first and second solutions the dopant material precipitates in the resulting solvent mixture. However, the stabiliser acts to prevent aggregation or flocculation of the solid particulates and limits their size, resulting in a fine suspension of particulates of the active species.

The suspension of the active species is stable, as shown in FIG. 3, which shows that sonication of fine suspensions of the tenofovir prodrug, tenofovir disoproxil fumerate, has no effect on their hydrodynamic diameter or size distribution.

In the method of the first aspect of the invention, the first and second solvents may be selected from alkanes, lower (C1-C10) alcohols, organic acids, amides, nitriles, cyclic hydrocarbons, halogenated alkanes, esters, aldehydes and ketones, ethers, volatile cyclic silicones and water. More preferably the first solvent system comprises at least one protic solvent and the second solvent system comprises at least one aprotic or non-polar solvent. The solvents may be selected from:
alkanes, for example heptane, n-hexane, iso-octane, decane, dodecane;
lower (C1-10) alcohols, such as methanol, ethanol, propanol, isopropanol, butanol, isobutanol, tertiary butanol, 1-pentanol;
organic acids, such as formic acid, acetic acid;
amides, such as formamide, N,N-dimethylformamide;
nitriles, such as acetonitrile;
cyclic hydrocarbons, such as toluene, xylene, cyclohexane;
halogenated alkanes, such as dichloromethane, dichloroethane, trichloromethane (chloroform), Fluorotrichloromethane, tatrachloroethane;
esters, such as ethyl acetate;
aldehydes and ketones, such as acetone, 2-butanon, 2-hexanone;
ethers, such as diethyl ether;
volative cyclic silicones, such as cyclomethicones containing from 4 to 6 silicon atoms, e.g. Dow Corning 245 Fluid and Dow Corning 345 Fluid, available from Dow Corning Inc.; and
water.

It is more preferable that the first solvent comprises a lower alcohol and the second solvent comprises a chlorinated solvent. It is most preferable that the first solvent comprises methanol and the second solvent comprises dichloromethane.

Methanol is a polar, protic solvent and is a suitable solvent for many active species. Dichloromethane is an aprotic solvent and is a suitable solvent for many stabilisers. Despite their different polarities, methanol and dichloromethane are fully miscible in any ratio, additionally, methanol and dichloromethane mixtures are suitable solvents for oils.

The first and second solvents may comprise further suitable solvents. Additional solvents may be used with the first and second solvents on the condition that they do not have a deleterious effect on the solubility of the active species, stabilisers or oils used in the method. The solvents that may be added to the first or second solvents are those that satisfy the requirements stipulated in the method. In other words, if using additional solvents, the first solvent must remain a solvent for the active species; the second solvent must remain a solvent for the stabiliser; the active species must remain insoluble in the mixed solvent resulting from the mixture of the first and second solvents; and the oil must remain soluble in the mixed solvent resulting from the mixing of the first and second solvents.

The mixtures of solvents for each of the first and second solvents are not limited to binary mixtures, but can include three or more components. Additional solvents can be present provided that they are miscible in the solvent mixture as a whole, do not solubilise the dopant material and do not render the oil insoluble in the solvent mixture. The additional solvents may be selected from the suitable solvents listed above.

It is preferable that the total concentration of the at least one active species in the first solution is between about 10 and 100 mg/mL, more preferably at least about 30 mg/mL, further preferably at least about 50 mg/mL, yet further preferably at least about 70 mg/mL and most preferably at least about 80 mg/mL. Additionally, it is preferable that the concentration of the stabilisers in the second solution is between about 1 and 30 mg/mL, more preferably at least about 5 mg/mL, further preferably at least about 10 mg/mL, yet further preferably at least about 15 mg/mL and most preferably at least about 20 mg/mL.

Alternatively, it may be preferred that the total concentration of the at least one active species in the first solution, that is kept within the range of about 30 and 100 mg/mL, is between about 50 and 100 mg/mL, more preferably between about 70 and 100 mg/mL and most preferably between about 80 and 100 mg/mL. As a further alternative, it may be preferred that the concentration of the stabilisers in the second solution, that is kept within the range of about 1 and 30 mg/mL, is between about 5 and 30 mg/mL, more preferably between about 10 and 30 mg/mL, further preferably between about 15 and 30 mg/mL and most preferably between about 20 and 30 mg/mL.

Varying the concentrations of the active species and stabilisers in the first and second solutions allows for the creation of particulates with different loadings of the active species without needing to alter the relative quantities of the first and second solutions or the total concentration of particulates in the resulting suspension. This is important as altering the ratios of the first and second solvents may alter the solubility of the active species and/or the oil in the resulting mixed solvent.

The volume ratio of the first and second solutions can be any volume ratio which satisfies the solubility criteria listed above (i.e. the precipitate particulates of active species must be insoluble in the mixed solvent resulting from the mixture of the first and second solvents; and the oil must be soluble in the mixed solvent resulting from the mixture of the first and second solvents). It is preferable that the volume ratio of the first and second solutions on mixing is between about 2:1 to 1:10, more preferably between about 1:1 and 1:6 and most preferably about 1:4.

The volume ratio of the first and second solutions is selected to ensure the precipitation of the active species in the resulting mixed solvent. The mixed solvent must also be able to solubilise the oil. The mixed solvent therefore contains solid particulates of active species and the oil in the liquid or dissolved state.

It is preferable for the first and second solutions to be agitated during the combining step. This agitation can simply be shaking manually, shaking on a plate shaker or other automated shaker, rolling on a tube roller, stirring with a magnetic stirrer, stirring with a mechanical stirrer, vortexing or sonication.

The methods used for the agitation while combining the first and second solutions are also suitable for mixing the oil into the suspension.

Many common laboratory methods are suitable to be employed to remove the solvents. A preferred method is lyophilisation (i.e. freeze-drying). Under this method, the oil-containing suspension is rapidly frozen using a cooling medium and the solvents removed by sublimation under reduced pressure. Preferably the cooling medium is liquid nitrogen (boiling point: −196° C.). Other possible cooling media include:
liquid air, (boiling point: −196° C.);
liquid ammonia, (boiling point: −33° C.);
liquefied noble gases, such as argon (boiling point: −186° C.);
liquefied halogenated hydrocarbon, such as trichloroethylene;
chlorofluorocarbons, such as Freon™; and
hydrocarbons, such as hexane, dimethylbutene, isoheptane, cumene.

The cooling media may be at its boiling point during the freezing process (as is the case for the liquid gases) or it may be cooled by external cooling means. The cooling medium may alternatively be an organic solvent with solid carbon dioxide. Alternatively, the cooling of the oil-containing suspension may be achieved through placing the oil-containing suspension in an environment held at a temperature below its freezing point, such as a freezer or freeze-dryer.

Preferably the reduced pressure is a high vacuum (e.g. <100 μBar). The conditions for freeze-drying are well known to those skilled in the art; the vacuum to be applied and the time taken should be such that effectively all of the solvent or mixture of solvents present is removed by sublimation. The freeze-drying step may be performed for up to around 72 hours, sometimes around 48 hours and preferably for less than 12 hours.

Lyophilisation may be used to remove the solvent from the liquid composition in bulk and then divided into preferred containers or vessels. Alternatively, lyophilisation may be used to remove the solvent from the liquid composition in the preferred containers, e.g. aseptic syringes, directly. It is preferred that the lyophilisation is performed so as to produce a sterile liquid composition.

The liquid compositions resulting from the method of the present invention are preferably substantially solvent-free. In the context of the present invention, the term “substantially solvent-free” means that the free solvent content of the compositions and drug preparations is less than 15%, preferably below 10%, more preferably below 5% and most preferably below 2%. Alternatively or additionally, the solvent content of the compositions is within the acceptable limits of industry-accepted regulatory guidelines for pharmaceuticals, such as the ICH Harmonised Guidelines. For the avoidance of doubt, throughout this specification, all percentages are percentages by weight unless otherwise specified.

Analogues of nucleotides and nucleosides are a class of compounds, many of which are pharmacologically active, whose structure is based on nucleotides and nucleosides, which are components of RNA and DNA. A nucleoside consists of a nucleobase and a ribose sugar. DNA uses four nucleobases: adenine, guanine, thymine and cytosine. Nucleobases can be defined as purines (adenine and guanine) or pyrimidines (cytosine, thymine and uracil) depending on their structure. RNA uses an additional nucleobase, uracil, in place of thymine.

The five naturally occurring nucleosides are adenosine, guanosine, 5-methyluridine, uridine and cytidine. A nucleotide consists of a nucleoside, with a phosphate group appended to the sugar (i.e. nucleoside monophosphate). The five naturally occurring nucleotides are adenine monophosphate, guanosine monophosphate, 5-methyluridine monophosphate, uridine monophosphate and cytidine monophosphate. As nucleosides and nucleotides are biological molecules, their analogues are of considerable interest for pharmaceutical applications. Suitable analogues of nucleosides may be analogues of any nucleoside; the nucleoside may be cyclic or acyclic. Suitable analogues of nucleotides may be analogues of any nucleotide; the nucleotide may be cyclic or acyclic. The structures of the ten naturally occurring nucleosides and nucleotides are provided below, for reference.

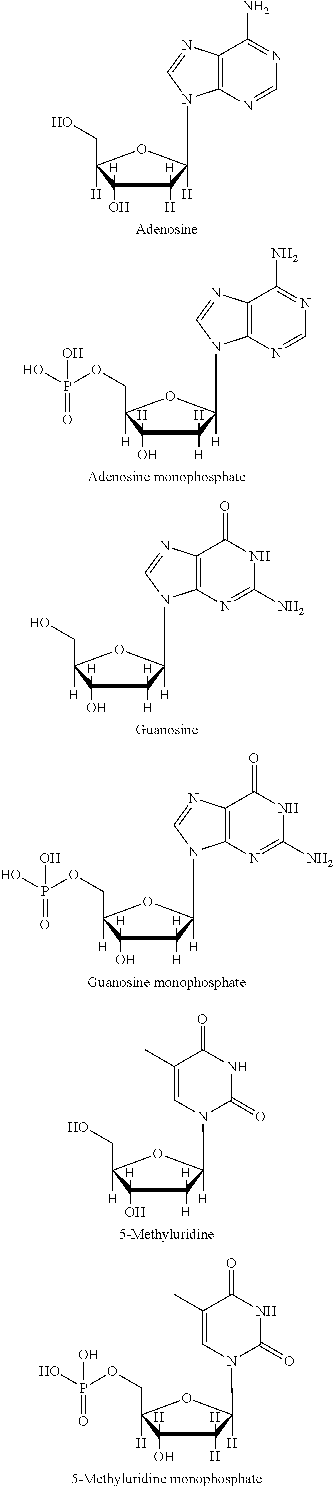

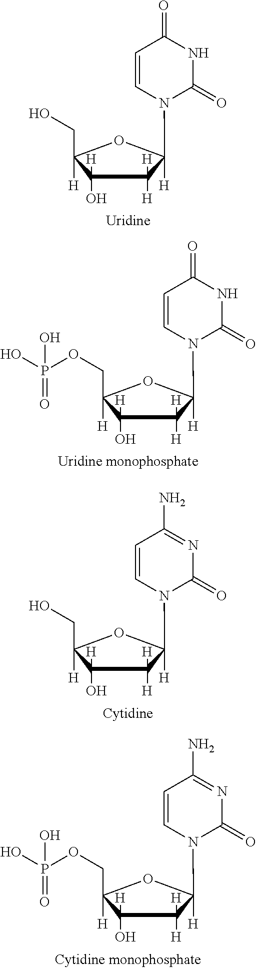

For the avoidance of any doubt, by analogue it is meant a structural analogue, also termed a chemical analogue. The structure of an analogue is similar to the eponymous compound, but different in certain respects. In other words, nucleoside analogues and nucleotide analogues are structurally similar to, but distinct from, nucleosides and nucleotides respectively. Modifications are made to the nucleobases and sugars for nucleoside analogues. Nucleotide analogues additionally feature modifications to the phosphate group. Common modifications include, but are not limited to: halogenation and N-conjugation of the nucleobase; halogenation, methylation, saturation, hydroxylation, dehydroxylation and ring-opening of the sugar; and the formation of esters and P-N bonds on the phosphate.

Well known classes of analogues of nucleotides and nucleosides are nucleotide analogue reverse-transcriptase inhibitors (NtRTIs) and nucleoside analogue reverse-transcriptase inhibitors (NRTIs). Analogues of nucleotides with activity against reverse-transcriptase are NtRTIs. Analogues of nucleosides with activity against reverse-transcriptase are NTRIs. NRTIs and NtRTIs are antiviral drugs effective against retroviruses, such as human immunodeficiency virus (HIV) or hepatitis B, acting to inhibit reverse-transcriptase. NRTIs may be analogues of adenosine, guanosine, 5-methyluridine, uridine and cytidine. NtRTIs may be analogues of adenine monophosphate, guanosine monophosphate, 5-methyluridine monophosphate, uridine monophosphate and cytidine monophosphate.

Preferably the nucleoside analogues are selected from adenosine analogues and guanosine analogues.

Preferably the nucleotide analogues are selected from adenosine monophosphate analogues and guanosine analogues. Optionally, the adenosine analogues may be selected from one or more of: adefovir prodrugs or tenofovir prodrugs.

Tenofovir prodrugs may be selected from tenofovir disoproxil, tenofovir alafenamide, their salts or combinations thereof.

The tenofovir prodrugs may be in the form of a pharmacologically acceptable salt. The formation of a salt, or the substitution of the counter-ion, may have effects on the pharmacokinetic and pharmacodynamic properties of the tenofovir prodrug. The tenofovir prodrug salt may be in the form of an organic salt such as acetate, oxalate, fumerate, citrate, succinate, tartrate, salicylate, benzoate, glycolate, methanesulfonate, ethanesulfonate, benzenesulfonate, p-toluenesulfonate, lactate, maleate, malonate, malate, isethionate, lactobionate, mandelate, p-coumarate, ferulate, sinapate, caffeate, chlorogenate, caftarate, coutarate, p- hydroxy benzoate, vanillate, syringate, 4-(4-phenoxybenzoyl) benzoate, gentisate, protocatechuate, gallate, lipoate, aspartate, orotate and the like. Alternatively, the tenofovir prodrug salt may be an inorganic salt such as hydrochloride, hydrobromide, sulfide, phosphate, nitrate, sulfamate and the like,

A preferred tenofovir disoproxil salt is tenofovir disoproxil fumerate (TDF).

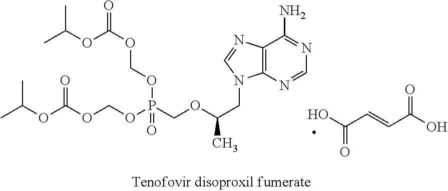 [SMILES: CC(C)OC(=O)OCOP(=O)(CO[C@H](C)Cn1cnc2c(N)ncnc21)OCOC(=O)OC(C)C ; O=C(O)/C=C/C(=O)O]

A preferred tenofovir alafenamide salt is tenofovor alafenamide fumerate (TAF).

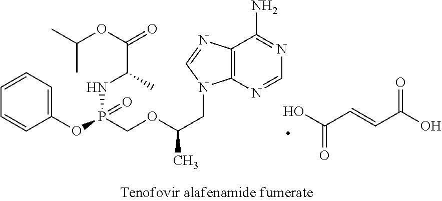

It is to be understood that multiple active species can be used in combination should they have solvents, stabilisers and oils which are mutually suitable, thereby forming particulates of active species comprising multiple active species. Alternatively, this results in a variety of particulates of active species with each variety of particulate comprising a single active species. Mutually suitable means that the solvents, stabilisers and oils selected are suitable for use in the method of the present application for the preparation of all of the active species under consideration. For example:
mutually suitable solvents means that the first solvent will dissolve all of the active species, the second solvent will dissolve all of the stabilisers, the active species will be insoluble in the mixed solvent and that the oil will dissolve in the mixed solvent;
mutually suitable stabilisers means that the stabilisers, in combination, will prevent the aggregation of any of the active species; and
mutually suitable oil means that none of the particulates of any of the active species will dissolve in the oil.

Advantageously, the one or more stabilisers are surfactants.

It is preferred that the one or more surfactants are selected from the class of anionic surfactants. Preferable anionic surfactants include sulfonate salts, more preferably sulfosuccinate salts.

Preferred anionic surfactants may be selected from dioctyl sulfosuccinate sodium salt, dioctyl sulfosuccinate potassium salt, dioctyl sulfosuccinate calcium salt and combinations thereof.

Dioctyl sulfosuccinate sodium salt (aka sodium docusate or AOT) is a particularly suitable anionic surfactant.

Without wishing to be bound by theory, it is thought that the anionic surfactant forms a salt with the tenofovir prodrug, or replaces the counter-ion of tenofovir prodrug salts, the resulting complex being insoluble in the mixed solvent. These complexes proceed to precipitate out of solution, forming particulates which are stabilised by any remaining stabiliser.

Additional stabilisers may be selected from anionic surfactants, non-ionic surfactants, cationic surfactants, zwitterionic surfactants and mixtures thereof.

The classes that the surfactants may be selected from, and some examples thereof, include:
anionic surfactants, such as alkylether sulfates; alkylether carboxylates; alkylbenzene sulfonates; alkylether phosphates; dialkyl sulfosuccinates; sarcosinates; alkyl sulfonates; soaps; alkyl sulfates; alkyl carboxylates; alkyl phosphates; paraffin sulfonates; secondary n-alkane sulfonates; alpha-olefin sulfonates; isethionate sulfonates; alginates;
cationic surfactants, such as fatty amine salts; fatty diamine salts; quaternary ammonium compounds; phosphonium surfactants; sulfonium surfactants; sulfonxonium surfactants;
zwitterionic surfactants, such as N-alkyl derivatives of amino acids (such as glycine, betaine, aminopropionic acid); imidazoline surfactants; amine oxides; amidobetaines; and
non-ionic surfactants, such as ethoxylated triglycerides; fatty alcohol ethoxylates; alkylphenol ethoxylates; fatty acid ethoxylates; fatty amide ethoxylates; fatty amine ethoxylates; sorbitan alkanoates; ethylated sorbitan alkanoates; alkyl ethoxylates; Pluronics™; alkyl polyglucosides; stearol ethoxylates; alkyl polyglycosides; sucrose fatty acid esters; propylene glycol monolaurate (Lauroglycol™ FCC); glyceryl monolinoleate (Maisine™ 35-1); corn oil PEG-6 esters (Labrafil™ M2125CS) and apricot kernel oil PEG-6 esters (Labrafil™ M1944CS).

Further stabilisers may be selected from polyoxyethylene (2) stearyl ether (Brij™ S2), propylene glycol monocaprylate (type II) (Capryol™ 90), propylene glycol monocaprylate (type I) (Capryol™ PGMC), propylene glycol dicaprylocaprate (Labrafac™ PG), apricot kernel oil PEG-6 esters (Labrafil™ M 1944 CS), corn oil PEG-6 esters (Labrafil™ M 2125 CS), caprylocaproyl polyoxyl-8 glycerides (Labrasol™) propylene glycol monolaurate (type II) (Lauroglycol™ 90), propylene glycol monolaurate (type I) (Lauroglycol™ FCC), glyceryl monolinoleate (Maisine™ 35-1), propylene glycol monopalmitostearate (Monosteol™), glycerol mono-oleate (Pecol™) polyglyceryl-3 diisostearate (Plurol™ Diisosteraque), polyglyceryl-6 dioleate (Plurol™ Olique), sorbitan oleate (Span™ 80), polyoxyethylenesorbitan monopalmitate (Tween™ 40), polyethylene glycol sorbitan monooleate (Tween™ 80) and combinations thereof.

It is to be understood that the choice of stabiliser, and additional stabiliser if used, may have an effect on the rate of release of the particulates of the at least one active species from the oil. It may additionally have an effect on the size and/or stability of the particulates in the mixed solvent and/or the oil.

It should be understood that combinations and mixtures of stabilisers may also be used in the method of the present invention.

The oil added to the particulate suspension may be selected from natural oils, mineral oils, synthetic oils, silicone oils and mixtures thereof. Suitable oils may have a boiling point higher than that of the solvents. Suitable oils may not solubilise the active species. The oil may be suitable for oral dosage as well as, or alternatively to, being suitable for parenteral administration.

Preferably the oil is a natural oil. Optionally, the natural oil is selected from peanut oil, soy bean oil, sesame oil, safflower oil, vegetable oil, avocado oil, rice bran oil, jojoba oil, Babassu oil, palm oil, coconut oil, castor oil, cotton seed oil, olive oil, flaxseed oil, rapeseed oil, acacia oil and mixtures thereof.

Preferably the oil is biocompatible as this would enable the liquid composition to be used in biological settings, for example as use in a medicament.

The selection of the oil should be such that the particulates of the at least one active species are insoluble (i.e. they remain solid when dispersed in the oil). The choice of oil may also have an effect on the rate of release of the particulates from the oil. The oil is contemplated as being capable of forming a depot of particulates. Optionally this depot can be placed in an intramuscular setting. Further optionally, this depot can be placed in a subcutaneous setting.

The oil should be a liquid as defined elsewhere in the specification. It is also contemplated that additional materials, which would ordinarily be solid under ambient conditions, can be mixed with the oil, on the proviso that the resulting mixture remains a liquid. The amount of the additional solid materials that may be added to the oil will be judged by the skilled person on a case-by-case basis as the nature of the oil and the additional solid materials being added will determine the ratio at which they can be mixed. It is preferred that the additional materials will dissolve into the oil, forming a homogenous solution (the particulates of the dopant material not-withstanding).

Additional materials may be oil-soluble polymers, which may be used to act as stabilisers for the liquid composition or as viscosity modifiers.

It is preferred that the additional materials are biocompatible as this would enable the liquid composition to be used in biological settings, for example as use in a medicament.

The oil may contain further oil-soluble materials, for example:
vitamins, such as esters of vitamin A (e.g. retinol palmitate or acetate), esters of vitamin E (e.g. tocopherol acetate or tocopherol linolate), vitamin B2, vitamin D6, vitamin F;
anti-inflammatory agents, such as bisabolol (also known as levomenol), glycerrethinic acid, stearyl glycerrhetinate;
lanolin and its derivatives; and
emollients, such as perhydrosqualene, perfluoropolyethers.

The one or more stabilisers for the method of the first aspect of the present invention may comprise AOT and optionally a further stabiliser. The further stabiliser may be propylene glycol monolaurate (type I) (Lauroglycol™ FCC) or glyceryl monolinoleate (Maisine™ 35-1). The further stabiliser may be propylene glycol monolaurate (type I) (Lauroglycol™ FCC). The oil may be selected from peanut oil, sesame oil, coconut oil and soybean oil. The oil may be sesame oil. The active species may be TDF.

The solids content of the suspension of stabilised particulates of the at least one active species suspended in the mixed solvent may comprise about 5-95% active species, preferably about 30-90% active species and most preferably about 60-85% active species by mass. The remainder of the mass of the stabilised particulates comprises stabilisers. If multiple stabilisers are used, the remainder of the mass may be divided equally between each of the stabilisers. Alternatively, a greater amount of one of the stabilisers may be used. The stabilised particulates may comprise about 80% active species, 10% of a first stabiliser and 10% of a second stabiliser. In one embodiment, the active species is TDF, the first stabiliser is AOT and the second stabiliser is propylene glycol monolaurate (type I) (Lauroglycol™ FCC) or glyceryl monolinoleate (Maisine™ 35-1), optionally the stabilised particulates comprise 80% TDF, 10% AOT and 10% of the second stabiliser. In another embodiment, the active species is TDF, the first stabiliser is AOT and the second stabiliser is propylene glycol monolaurate (type I) (Lauroglycol™ FCC), optionally the stabilised particulates comprise 80% TDF, 10% AOT and 10% propylene glycol monolaurate (type I) (Lauroglycol™ FCC).

The suspension of stabilised particulates may be mixed with the oil to achieve a predetermined loading of active species after removal of the mixed solvents. The loading may be at least 1 mg/mL, preferably at least 10 mg/mL, more preferably at least 40 mg/mL and most preferably at least 60 mg/mL. Alternatively, the loading may be about 1-50 mg/mL, about 5-25 mg/mL, about 10-20 mg/mL or about 15 mg/mL. In an embodiment, the active species is TDF.

### Second Aspect

A second aspect of the present invention provides a liquid composition comprising stabilised particulates of at least one active species in an oil, the active species being selected from nucleoside analogues and nucleotide analogues.

The second aspect of the present invention also contemplates a liquid composition comprising stabilised particulates of at least one active species in an oil which has been produced using the method of the first aspect of the present invention.

In said liquid compositions the nucleoside analogues may be selected from adenosine analogues and guanosine analogues. Optionally the nucleotide analogues are selected from adenosine phosphate analogues and guanosine phosphate analogues.

Preferably, the adenosine phosphate analogues may be selected from one or more of: adefovir prodrugs or tenofovir prodrugs.

The tenofovir prodrug may be selected from tenofovir disoproxil, tenofovir alafenamide, their salts or combinations thereof. A preferred tenofovir disoproxil salt is tenofovir disoproxil fumerate (TDF). A preferred tenofovir alafenamide salt is tenofovor alafenamide fumerate (TAF).

The oil may be selected from natural oils, mineral oils, synthetic oils, silicone oils and mixtures thereof, on the proviso that the oil does not solubilise the one or more tenofovir prodrugs.

Suitable natural oils may be selected from peanut oil, soy bean oil, sesame oil, safflower oil, vegetable oil, avocado oil, rice bran oil, jojoba oil, Babassu oil, palm oil, coconut oil, castor oil, cotton seed oil, olive oil, flaxseed oil, rapeseed oil, acacia oil and mixtures thereof. It is further preferred that the oil is biocompatible.

The z-average diameters of the particulates of the at least one active species are determined by any suitable means which would be known by the skilled person. A preferred method is dynamic light scattering (DLS), performed on the particulates in the mixed solvent system prior to the addition of the oil. The particularly preferred method of particle sizing for the particulates of the at least one active species is DLS employing a Zetasizer Nano S instrument (manufactured by Malvern Instruments UK). Specifically, the Malvern Instruments Nano S uses a red (633 nm) 4 mW Helium-Neon laser to illuminate a standard optical quality UV cuvette containing the suspension of particulates in the mixed solvent system. The z-average diameters quoted in this application are those obtained with that apparatus using the standard protocol provided by the instrument manufacturer.

The particulates may comprise about 5-95% active species, preferably about 30-90% active species and most preferably about 60-85% active species by mass. The remainder of the particulates of the at least one active species are comprised of stabilisers.

As explained above, it is preferable for the composition of the particulates of the active species (i.e. the wt % of the particulates which is the active species) to be altered by varying the relative concentrations of the first and second solutions such that the volumes of each of the first and second solutions which are mixed and the total concentration of the particulates of the at least one active species are consistent between batches. In other words, to make particulates with different loadings of the active species the concentrations of the first and second solutions are varied while keeping the total volumes of each solution and the total mass of active species plus stabiliser constant.

Preferably the concentration of the particulates of the at least one active species in the liquid composition is at least 1 mg/mL, preferably at least 10 mg/mL, more preferably at least 40 mg/mL and most preferably at least 60 mg/mL.

The liquid composition may have a loading of active species of at least 1 mg/mL, preferably at least 10 mg/mL, more preferably at least 40 mg/mL and most preferably at least 60 mg/mL. Alternatively, the loading may be about 1-50 mg/mL, about 5-25 mg/mL, about 10-20 mg/mL or about 15 mg/mL. In embodiments, the active species is TDF.

Alternatively the concentration of the particulates of the at least one active species in the liquid composition is between about 1 mg/mL and about 100 mg/mL, preferably between about 10 mg/mL and about 80 mg/mL and most preferably between about 20 mg/mL and about 60 mg/mL.

The concentration of the particulates of the at least one active species in the liquid composition can be finely tuned simply by altering the volume of oil added prior to the freeze drying process. High concentrations of the particulates of the at least one active species result in a thick suspension, which can nonetheless be passed through a needle using a syringe. Therefore even at high concentrations of the particulates the liquid composition has material properties suitable for that of an injectable. Should a biocompatible oil be selected the liquid composition will also be biocompatible, and thus suitable for biological applications such as intramuscular injection.

Low concentrations of the particulates of the at least one active species in the liquid composition may find use in more sensitive in vitro assays.

Liquid compositions of any viscosity (i.e. including those too thick to be passed through a needle using a syringe and thus unsuitable for use as an injectable), can be suitable for oral administration. Oral administration may be as a liquid syrup. Alternatively, the liquid composition can be packed into a capsule. Optionally, the capsule can be a gel-capsule.

Optionally, should multiple liquid compositions comprising different active species use mutually suitable oils the liquid compositions may be mixed, thereby resulting in a liquid composition comprising a variety of particulates, each variety of particulate comprising a single active species or combination of active species.

The oil comprising the liquid composition according to the second aspect of the present invention may be selected from peanut oil, sesame oil, coconut oil and soybean oil. The oil may be sesame oil. If multiple stabilisers are used, the remainder of the mass may be divided equally between each of the stabilisers. Alternatively, a greater amount of one of the stabilisers may be used. The stabilised particulates may comprise about 80% active species, 10% of a first stabiliser and 10% of a second stabiliser.

In embodiments, the active species is TDF and the oil is selected from peanut oil, sesame oil, coconut oil and soybean oil, preferably sesame oil. Optionally, the loading of active species in the liquid compositions is at least 1 mg/mL, preferably at least 10 mg/mL, more preferably at least 40 mg/mL and most preferably at least 60 mg/mL.

Alternatively, the loading of the active species may be about 1-50 mg/mL, about 5-25 mg/mL, about 10-20 mg/mL or about 15 mg/mL.

### Third Aspect

A third aspect of the present invention provides a pharmaceutical or veterinary composition in liquid dosage form comprising a liquid composition according the second aspect of the present invention. The pharmaceutical or veterinary composition optionally includes one or more additional (pharmaceutically acceptable) excipients.

Possible excipients include any known to one skilled in the art, and may be drawn from the classes of: fillers, diluents, flavours, colourants, preservatives, sweeteners and vehicles.

It will be appreciated that different pharmaceutical or veterinary compositions of the invention may be obtained by conventional procedures, using conventional pharmaceutical excipients, well known in the art.

The pharmaceutical or veterinary composition may further comprise additional pharmacologically active compounds. The pharmaceutical or veterinary composition may be a combination product including further pharmacologically active compounds.

Further pharmacologically active compounds includes further nucleoside analogues and nucleotide analogues, which may or may not be suitable for formulating into particulates of active species suspended in oil.

Such further pharmacologically active compounds may be dissolved into the oil or present as additional particulates suspended in the oil phase. The additional particulates may be made by the method of the present invention or any other suitable method, for example by spray drying or emulsion routes. Preferably, the further pharmacologically active compounds have a synergistic effect with the active species. A particularly preferable pharmaceutical or veterinary composition is one which is suitable for parenteral administration, for example by either intramuscular or sub-cutaneous injection. More preferably, such an injection would form a depot of the active species at the injection site, which would gradually release the active species into the area surrounding the injection site. The gradual release of the active species may be over a predetermined period of time. Preferably, the release of the active species maintains a set concentration range of the active species in the area surrounding the injection site. Optionally the injection site is an intramuscular or subcutaneous site and the area surrounding the injection site is biological tissue. The set concentration range to be maintained may be a therapeutically effective concentration range. The area surrounding the injection site may be considered to extend to the entirety of the biological system in question, or even to the entirety of the organism.

The pharmaceutical or veterinary composition may be provided in a suitable aseptic container. Preferably the container is a sealed vial. More preferably, the container is a prefilled syringe.

Alternatively, the pharmaceutical or veterinary composition is in a form suitable to be administered orally. A suitable form for the oral administration of the pharmaceutical or veterinary composition may be a filled capsule or a syrup. A preferable form for the pharmaceutical or veterinary composition to be administered orally may be a gel capsule. An alternative form for the pharmaceutical or veterinary composition to be administered orally may be a syrup.

The bioavailability of orally taken active species is often enhanced by the simultaneous consumption of fats (for example, the uptake of tenofovir disoproxil fumerate in the gut). Formulating the active species as particulates dispersed in an oil enhances their oral bioavailability. This enhancement does not require any further action on the part of the patient (i.e. administering the composition with a fatty meal), thereby ameliorating the issue of patient compliance. This improved oral bioavailability may also result in lower dosing regimens, thereby reducing adverse side effects and lowering long-term toxicity.

### Fourth Aspect

A fourth aspect of the present invention provides a liquid composition according to the second aspect of the present invention, or a pharmaceutical or veterinary composition according to the fourth aspect of the present invention, for use as a medicament.

### Fifth Aspect

A fifth aspect of the present invention concerns a liquid composition according to the second aspect of the present invention, or a pharmaceutical or veterinary composition according to the fourth aspect of the present invention, for use in the treatment and/or prevention of viral infections.

It is to be appreciated that references to “preventing” or “prevention” relate to prophylactic treatment and includes preventing or delaying the appearance of clinical symptoms of the state, disorder or condition developing in a human that may be afflicted with or predisposed to the state, disorder or condition but does not yet experience or display clinical or subclinical symptoms of the state, disorder or condition.

It will be appreciated that references to “treatment” or “treating” of a state, disorder or condition includes: (1) inhibiting the state, disorder or condition, i.e., arresting, reducing or delaying the development of the disease or a relapse thereof (in case of maintenance treatment) or at least one clinical or subclinical symptom thereof, or (2) relieving or attenuating the disease, i.e. causing regression of the state, disorder or condition or at least one of its clinical or subclinical symptoms.

Optionally, the viral infection may be caused by the human immunodeficiency virus (HIV).

### Sixth Aspect

A sixth aspect of the present invention is a method of treating and/or preventing an infection, the method comprising administering a therapeutically effective amount of a liquid composition according to the second aspect of the present invention, or a pharmaceutical or veterinary composition according to the fourth aspect of the present invention, to a patient suffering from or at risk of a viral infection.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the patient to be treated.

Preferably, the viral infection is caused by HIV.

The administration of a therapeutically effective amount of the liquid composition or the pharmaceutical or veterinary composition may result in the formation of a depot of the particulates of the at least one active species suspended in the oil. The active species may be released from the depot gradually.

It is preferred that the administration of a therapeutically effective amount of the liquid composition or the pharmaceutical or veterinary composition results in a therapeutically effective concentration of the active species being maintained for at least 24 hours, preferably at least 36 hours, preferably at least 48 hours, more preferably at least 72 hours and most preferably 96 hours. Alternatively, the therapeutically effective concentration of the active species is maintained for between about 12 hours and about 96 hours, preferably between about 24 hours and about 96 hours, more preferably between about 48 hours and about 96 hours and most preferably between about 72 hours and about 96 hours.

In other words, the pharmacokinetic profile of the active species is significantly enhanced by incorporation into the liquid composition described above. FIG. 1 displays projected pharmacokinetic profiles of an active species, such as TDF, in an animal model. In the upper plot the plasma concentration of the active species increases rapidly initially, but then decreases gradually, maintaining a therapeutically effective concentration over a long time period. In the lower plot the concentration of active species increases initially, but soon approaches a steady state as the rate of release of the active species from the oil approximates the rate of removal of the active species from the blood plasma by metabolism and excretion. In this way, a set concentration, which may be a therapeutically effective concentration, can be maintained over long time periods.

It will be appreciated that the pharmacokinetics of the active species may fall between these scenarios, or may even change from one to the other over time. Numerous factors determine which regime the release of the active species predominates, such as formulation, depot release rate, metabolism and clearance. Additionally, these factors may be mutually exclusive, but may also have a degree of interplay between them.

It is thought that the rate of the rate of release of the particulates of the active species can be controlled by varying the initial concentration and the size of the particulates of the active species, the identity and quantity of the stabilisers, the identity of the oil and the identity and quantity of any additional materials included in the oil. For example, particulates of active species will diffuse more slowly through more viscous oils, thereby slowing the rate of release of the active species.

A gradual release of the at least one active species will result in the administration of a therapeutically effective amount of the liquid composition or the pharmaceutical or veterinary composition taking place with a reduced frequency, thereby improving patient compliance and efficacy of the method.

Alternatively, the administration of a therapeutically effective amount of the liquid composition or the pharmaceutical or veterinary composition may result in improved oral bioavailability of the active species in comparison to administration of standard formulations of the active species. This improved oral bioavailability may result in reduced dosing, thereby ameliorating adverse side effects and long-term toxicity.

### Seventh Aspect

A seventh aspect of the present invention concerns a liquid composition according to the second aspect of the present invention, or a pharmaceutical or veterinary composition according to the fourth aspect of the present invention, for use in the treatment of cancers.

### Eighth Aspect

An eighth aspect of the present invention is a method of treating cancer, the method comprising administering a therapeutically effective amount of a liquid composition according to the second aspect of the present invention, or a pharmaceutical or veterinary composition according to the fourth aspect of the present invention, to a patient suffering from cancer.

The administration of a therapeutically effective amount of the liquid composition or the pharmaceutical or veterinary composition may result in the formation of a depot of the particulates of the at least one active species suspended in the oil. The active species may be released from the depot gradually.

It is preferred that the administration of a therapeutically effective amount of the liquid composition or the pharmaceutical or veterinary composition results in a therapeutically effective concentration of the active species being maintained for at least 24 hours, preferably at least 36 hours, preferably at least 48 hours, more preferably at least 72 hours and most preferably 96 hours. Alternatively, the therapeutically effective concentration of the active species is maintained for between about 12 hours and about 96 hours, preferably between about 24 hours and about 96 hours, more preferably between about 48 hours and about 96 hours and most preferably between about 72 hours and about 96 hours.

A gradual release of the at least one active species will result in the administration of a therapeutically effective amount of the liquid composition or the pharmaceutical or veterinary composition taking place with a reduced frequency, thereby improving patient compliance and efficacy of the method.

Alternatively, the administration of a therapeutically effective amount of the liquid composition or the pharmaceutical or veterinary composition may result in improved oral bioavailability of the active species in comparison to administration of standard formulations of the active species. This improved oral bioavailability may result in reduced dosing, thereby ameliorating adverse side effects and long-term toxicity.

### DESCRIPTION OF THE DRAWINGS

FIG. 1 shows projected pharmacokinetic profiles displaying the effects of a slow release of an active species from a depot in an animal model. The upper plot shows the case where the concentration of the active species peaks and then subsides gradually, while the lower plot shows the case where the concentration of the active species remains level from the time of administration.

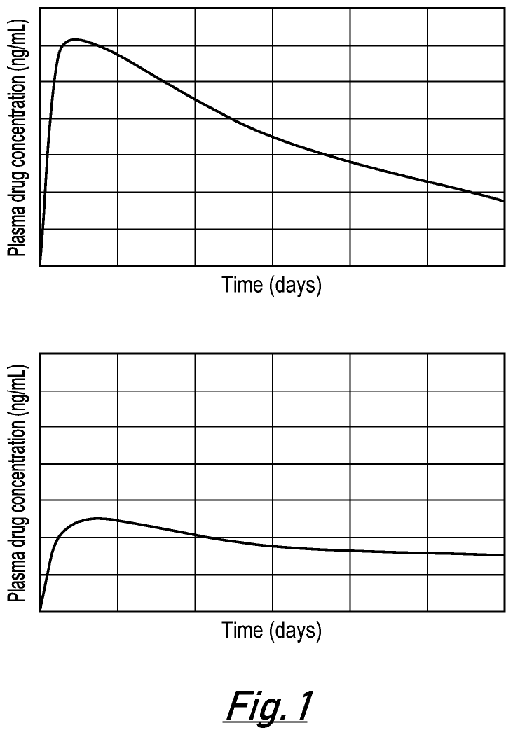

FIG. 2 is a graph of the distribution of the z-average hydrodynamic diameters of the TDF particulates of Example 2 with 60, 70 and 80 wt % loadings of TDF in dichloromethane as determined by DLS measurements.

FIG. 3 is a graph of the distribution of the z-average hydrodynamic diameters of the TDF particulates of Example 2 with a 60 wt % loading of TDF before and after sonication.

FIGS. 4a and 4b show measured pharmacokinetic profiles for a number of liquid compositions comprising stabilised particles of TDF in various oils following a single oral dose. Each graph also shows the measured pharmacokinetic profile for an unformulated control for comparison.

FIG. 5 shows a measured steady-state pharmacokinetic profile for a liquid composition comprising stabilised particles of TDF in Sesame oil following multiple oral dose over 42 hours. The graph also shows the measured steady-state pharmacokinetic profile for an unformulated control for comparison.

### EXAMPLES

### Particle size measurement

The hydrodynamic diameters and polydispersity indices of the particulates of dopant material were measured following the precipitation of the dopant material but prior to the addition of the oil. The analysis was performed using DLS, specifically a Zetasizer Nano S (Malvern Instruments, Malvern, UK) with the following parameters:
The particle type was set as nanoparticles with a refractive index of 1.330 and Absorption of 0.010
The dispersant was dichloromethane with a viscosity of 0.4130 and refractive index of 1.424
The sample temperature was 25° C.
A quartz cuvette with a path length of 10 mm was used
The measurement angle was 172°
3 measurements were taken per sample
The number of runs per measurement was selected automatically.

### Lyophilisation

The solvents were removed by lyophilisation. The mixture to be dried was rapidly frozen using liquid nitrogen and connected to a VirTis Benchtop K freeze dryer. The condenser was set to −100° C., the pressure was maintained ≤20 μbar and the sample dried for 48 hours.

The examples relate to the formation of liquid compositions comprising tenofovir disoproxil fumerate (TDF).

### Example 1—Solvent and Stabiliser Selection

TDF is a water-soluble prodrug of tenofovir and is known to be soluble in methanol and, for the purposes of this invention, it was found to be soluble up to 80 mg/mL in this solvent. Therefore methanol was used for the first solvent. TDF is not soluble in dichloromethane, which was used as the second solvent.

A range of stabilisers were then tested for solubility in dichloromethane, mixtures of dichloromethane and methanol and a variety of potential oils. Of the stabilisers tested, five were found to be suitable and are listed in Table 1 below along with their molecular weights and HLB values.

TABLE 1

| Stabiliser | Molecular weight (Da) | HLB Value |
|---|---|---|
| AOT | 444.56 | 10.5 |
| Lauroglycol ™ FCC | 258.40 | 5 |
| Maisine ™ 35-1 | 354.52 | 4 |
| Labrafil ™ M2125CS | Variable | 4 |
| Labrafil ™ M1944CS | Variable | 4 |

The ability of these stabilisers to prevent the aggregation of the TDF particulates was investigated, as shown in Table 2 below, when used alone or in binary (50:50 by mass) combinations.

TABLE 2

| Formulation ID | Stabiliser A | Stabiliser B |
|---|---|---|
| SC 1 | AOT | — |
| SC 2 | AOT | Lauroglycol ™ FCC |
| SC 3 | AOT | Labrafil ™ M2125CS |
| SC 4 | AOT | Maisine ™ 35-1 |
| SC 5 | AOT | Labrafil ™ M1944CS |

### Example 2—Synthesis of TDF particulates

The components were selected according to the above results: SC 4 was used as the stabiliser, the first solvent was methanol and the second solvent was dichloromethane.

The procedure to synthesise TDF particulates was as follows.

Stock solutions of TDF and SC 4 with specific concentrations (see Table 3 below) were made up by stirring the mass of TDF and stabilisers required to create the targeted concentrations in methanol and dichloromethane respectively. Stirring was achieved using a magnetic stirrer bar and magnetic hot-plate at room temperature.

TABLE 3

| | | Concen- | | Total volume |
|---|---|---|---|---|
| | Concen- | tration | Total volume | of SC 4 in |
| TDF | tration | of SC 4 in | of TDF in | dichloro- |
| loading of | of TDF in | dichloro- | methanol | methane |
| particulates | methanol | methane | added | added |
| (wt %) | (mg/mL) | (mg/mL) | (μL) | (μL) |
| 50 | 50 | 12.5 | 200 | 800 |
| 60 | 60 | 10 | 200 | 800 |
| 70 | 70 | 7.5 | 200 | 800 |
| 80 | 80 | 5 | 200 | 800 |

800 μL of the stabiliser-containing dichloromethane solution was pipetted into a 4 mL volume glass vial. 200 μL of TDF-containing methanol solution was rapidly pipetted into the same vial, which was then sealed and briefly shaken. A white, hazy suspension formed instantaneously. The 4:1 ratio of the solutions was kept constant as it was found to be effective for the formation of TDF particulates. The TDF loading of the particulates was altered by changing the concentration of the methanol and dichloromethane solutions prior to mixing. It should be noted that this approach allows for different volumes of the TDF particulate suspensions to be produced simply by increasing the volumes added, so long as the 4:1 ratio of dichloromethane to methanol is maintained, this allows for fine control of the concentration of the TDF particles dispersed in the oil of the liquid composition.

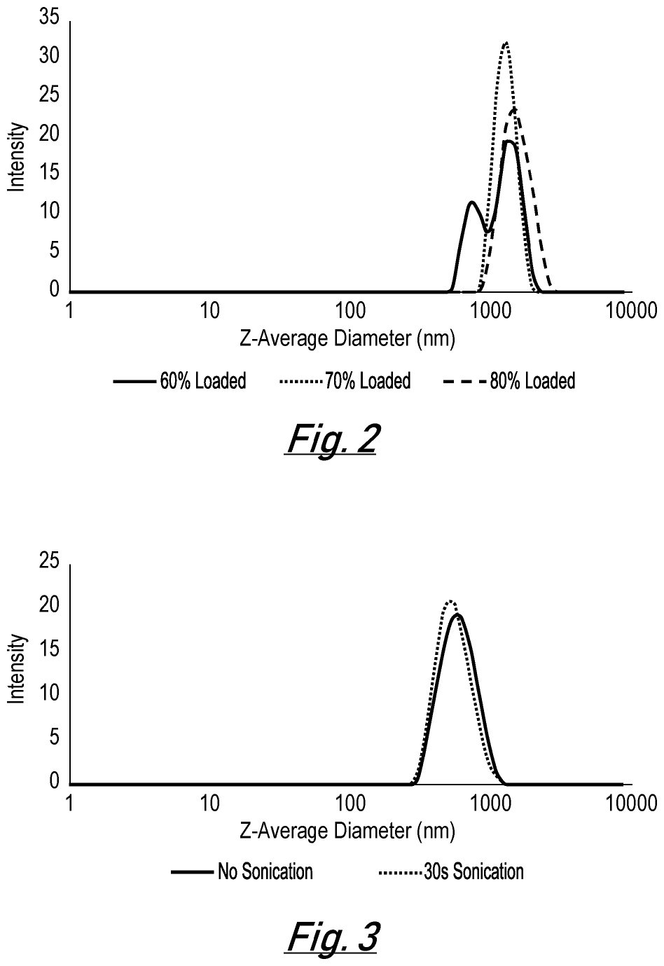

The hydrodynamic diameters and polydispersity indices of the TDF nanoparticles were determined (see Table 4 below and FIG. 2) and it was found that the diameter of the TDF particulates increased with TDF loading. Without wishing to be bound by theory, it was hypothesised that this is due to the samples with higher TDF loading having a reduced amount of stabiliser, reducing the stabilisers ability to prevent particle growth upon precipitation.

TABLE 4

| | Z-average | |
|---|---|---|
| TDF | hydrodynamic | |
| loading | diameter | Polydispersity |
| (wt %) | (nm) | index |
| 60 | 1125 | 0.172 |
| 70 | 1236 | 0.395 |
| 80 | 1554 | 0.114 |

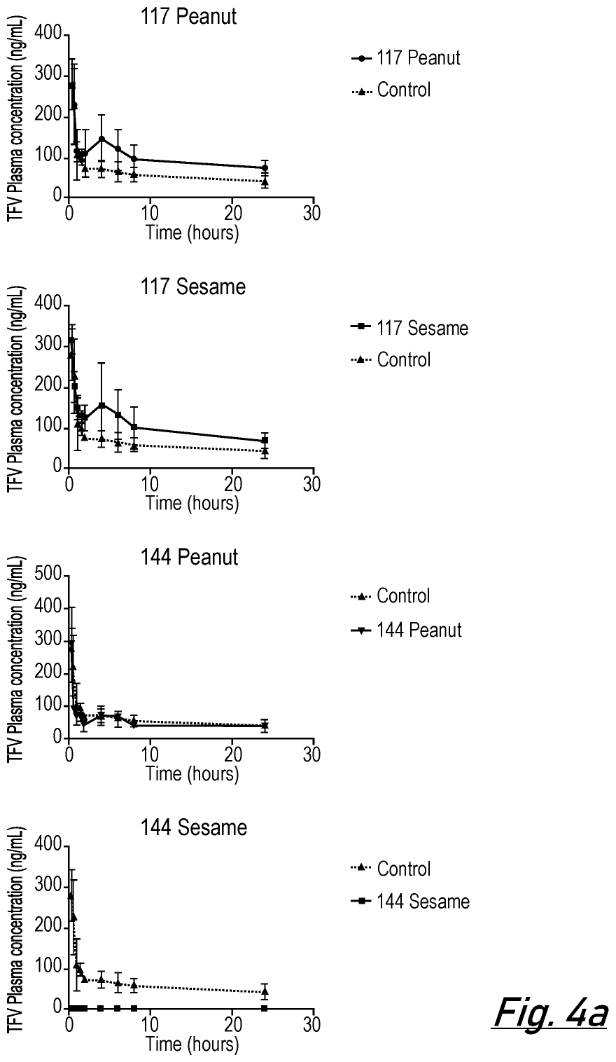

The TDF particulates were subjected to sonication and their hydrodynamic diameters and polydispersity indices re-measured to determine the stability of the TDF particulates. Sonication had no effect on the TDF particulate's size (see FIG. 3), indicating that the TDF particulates are stable in the 4:1 dichloromethane:methanol solvent system.

### Example 3—Synthesis of the Liquid Composition comprising TDF Particulates

A selection of biocompatible natural oils were used to form liquid compositions. These oils were selected as the TDF particulates were insoluble in them and the oils could be dissolved into the mixed solvent system. The oils used were peanut oil, soy bean oil, sesame oil and safflower oil.

The procedure to synthesise the liquid compositions comprising TDF particulates was as follows.

The oil was added to the suspension via pipette and mixed by vortexing to form a homogenous solution with the mixed solvent system. This mixture was then dried by lyophilisation as described above. The methanol and dichloromethane solvents were removed by this process, leaving TDF particulates suspended in an oil. The oil phase was initially frozen, but returned to liquid form after standing at room temperature for approximately 5 minutes.

The concentration of the TDF particulates in the oil was easily altered by varying the volumes of the suspension and oil prior to lyophilisation. High concentrations of up to 60 mg/mL were achieved with these TDF particulates, forming a suspension which was thick, but still passable through a 25 G needle despite its high viscosity, demonstrating that the liquid composition at least meets the physical requirements for an injectable composition.

TABLE 5

| Total sample volume | Volume of TDF | | Volume | Final concentration | |
|---|---|---|---|---|---|
| and possible | particulate | | of | of TDF particulates | |
| application | suspension | | oil | dispersed in oil | |
| 2 mL-in vitro assay | 100 | μL | 2 mL | 1 | mg/mL |
| 1 mL-in vivo study | 3 | mL | 1 mL | 60 | mg/mL |
| 5 mL-physical | 5 | mL | 5 mL | 20 | mg/mL |
| characterisation | | | | | |

Larger quantities of the liquid composition were synthesised by simply using more of the TDF suspension and the oil, additionally lower concentrations were produced in a facile manner by dilution with more oil (see Table 5 above). These low concentrations may be useful for more sensitive applications, such as in vitro assays.

### Example 4—Range of Liquid Compositions comprising TDF particulates

The methods outlined in Examples 2 and 3 were used to make a range of formulations of TDF particulates with the full ranges of stabilisers and oils outlined above. The fact that all of these were successful highlights the flexibility of the method of the present invention. Using the 5 stabilisers, 4 oils and 4 TDF loadings provided above, 80 formulations were successfully synthesised (see Table 6 below).

TABLE 6

| | TDF loading | Success of dispersion in the Oil | | | |
|---|---|---|---|---|---|
| Stabiliser | (wt %) | Peanut | Sesame | Soy Bean | Safflower |
| SC 1 | 50 | Y | Y | Y | Y |
| | 60 | Y | Y | Y | Y |
| | 70 | Y | Y | Y | Y |
| | 80 | Y | Y | Y | Y |
| SC 2 | 50 | Y | Y | Y | Y |
| | 60 | Y | Y | Y | Y |
| | 70 | Y | Y | Y | Y |
| | 80 | Y | Y | Y | Y |
| SC 3 | 50 | Y | Y | Y | Y |
| | 60 | Y | Y | Y | Y |
| | 70 | Y | Y | Y | Y |
| | 80 | Y | Y | Y | Y |
| SC 4 | 50 | Y | Y | Y | Y |
| | 60 | Y | Y | Y | Y |
| | 70 | Y | Y | Y | Y |
| | 80 | Y | Y | Y | Y |
| SC 5 | 50 | Y | Y | Y | Y |
| | 60 | Y | Y | Y | Y |
| | 70 | Y | Y | Y | Y |
| | 80 | Y | Y | Y | Y |

### Example 5—Additional Stabilisers

Following the procedure described in Example 2, the following combinations of stabilisers were used to form TDF particulates (see table 7 below).

TABLE 7

| Stabiliser A | Stabiliser B | Successful Precipitation |
|---|---|---|
| AOT | Brij ™ S2 | Yes |
| AOT | Capryol ™ 90 | Yes |
| AOT | Capryol ™ PGMC | Yes |
| AOT | Labrafac ™ PG | Yes |
| AOT | Labrafil ™ M 1944 CS | Yes |
| AOT | Labrafil ™ M 2125 CS | Yes |
| AOT | Labrasol ™ | Yes |
| AOT | Lauroglycol ™ 90 | Yes |
| AOT | Lauroglycol ™ FCC | Yes |
| AOT | Maisine ™ 35-1 | Yes |
| AOT | Monosteol ™ | Yes |
| AOT | Pecol ™ | Yes |
| AOT | Plurol ™ diisosteraque | Yes |
| AOT | Plurol ™ Olique | Yes |
| AOT | Span ™ 80 | Yes |
| AOT | Tween ™ 40 | Yes |
| AOT | Tween ™ 80 | Yes |

All of the stabilisers were found to be effective when used in combination with AOT.

### Example 6—In vivo Oral Bioavailability from a Single Dose

Adult male Wistar rats (˜300 g) were divided into 9 groups (3 rats per group, 8 TDF particulate dispersions and an oral control of 10 wt% TDF in an acacia vehicle). Following habituation (7 days) the rats received the single dose of a TDF particulate dispersion or unformulated TDF control via oral gavage (15 mg/Kg of TDF, 1 mL/kg). Food and water was provided ad libitum throughout the procedure. Blood samples were collected (250 μl) post dosing from the tail vein over 24 hours.

The formulations used in this Example comprised a suspension of 80 wt % TDF, 10 wt % AOT and 10 wt % of either Lauroglycol FCC or Maisine 35-1 at a concentration of 20 mg total solids per millilitre. 1 mL of each suspension was then mixed with 1.067 mL of oil and then lyophilised to give a particulate concentration of 18.75 mg/mL in the oil, equivalent to 15 mg/mL of TDF.

The weight of each rat was determined prior to sampling and monitored as an estimation of wellbeing throughout the experiment. At the point of termination, rats were sacrificed using a rising gradient of CO₂ followed by cervical dislocation.

Bioanalysis was performed on a TSQ Endura (Thermoscientific) using a validated assay for TDF in plasma. A calibration curve of TDF was prepared in rat plasma via serial dilution, ranging from 1.9 to 500 ng/ml. Extraction was performed using solid phase extraction. Linearity was assessed by three independent preparations of the standard curve. Maximum allowed deviation of standards was set at 15% of the stated value, excluding the LLOQ where deviation was set at no more than 20%.

Inter- and intra-assay accuracy and precision was assessed by preparation of three concentrations (in the range of the standard curve 10, 200 and 400 ng/mL) with each preparation in triplicate. Acceptable deviation from the mean values was defined as 15% of the stated concentration (except the lower concentration, where deviation was <20%).

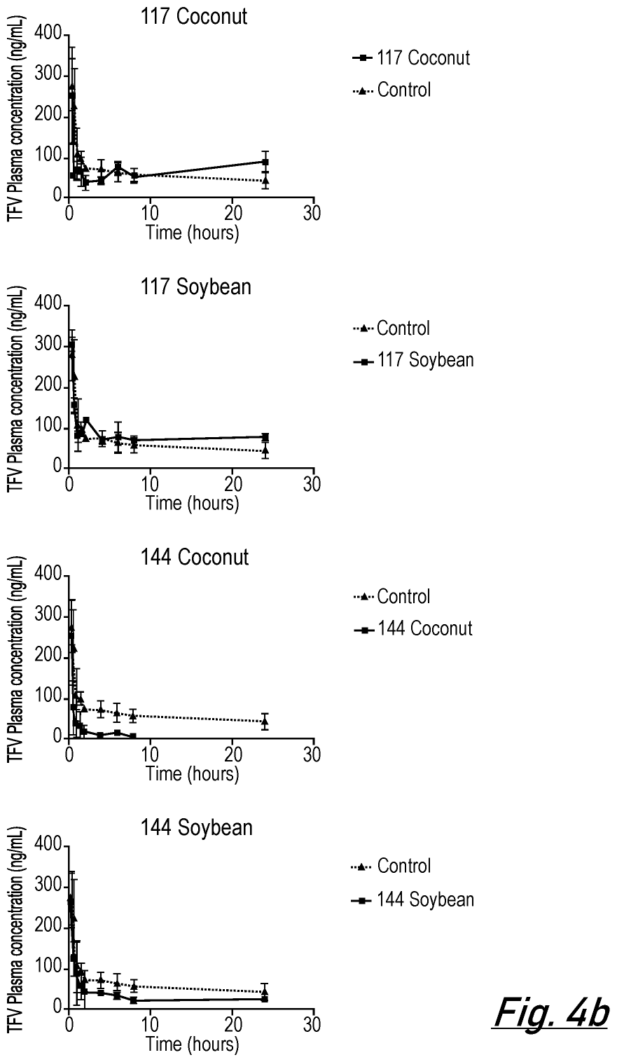

Plasma concentrations of TDF were then plotted using Prism (v7.0a). Pharmacokinetic parameters (Cₘₐₓ, Tₘₐₓ and AUC) were calculated using the PKsolver plugin and are displayed in Table 8 (standard deviations for each measurement shown in brackets) and FIGS. 4a and 4b (standard deviations for each measurement shown as error bars).

TABLE 8

| | | Cₘₐₓ | Cₘᵢₙ | AUC |
|---|---|---|---|---|
| Formulation | Stabilisers | (ng/mL) | (ng/mL) | (ng/h/mL) |
| Control | 10% Acacia | 314 (36.3) | 67.3 (19.0) | 2465 (759.3) |
| 117 Peanut | AOT + Lauroglycol FCC | 294 (70) | 43 (19.6) | 1492 (270.1) |
| 117 | AOT + Lauroglycol FCC | 258 (47.5) | 90 (29.8) | 1833 (821.0) |
| Sesame | | | | |
| 117 | AOT + Lauroglycol FCC | 253 (115.6) | 105 (21.3) | 1780 (319.6) |
| Coconut | | | | |
| 117 Soy | AOT + Lauroglycol FCC | 304 (18.9) | 77 (8.4) | 1909 (83.6) |
| Bean | | | | |
| 144 Peanut | AOT + Maisine 35-1 | 292 (115.6) | 39 (2.6) | 1044 (402.0) |
| 144 | AOT + Maisine 35-1 | 0 (0.0) | 0 (0.0) | 0 (0.0) |
| Sesame | | | | |
| 144 Soy | AOT + Maisine 35-1 | 268 (66.5) | 26.3 (4.3) | 811 (51.5) |
| Bean | | | | |
| 144 | AOT + Maisine 35-1 | 252 (18.7) | 18.2 (18.9) | 405 (113.2) |
| Coconut | | | | |

As can be seen from the results, the in vivo data for the single dose screen of the 8 TDF particulate dispersions demonstrated similar plasma exposure to that observed with the control over a 24 hour period.

### Example 7—In vivo Oral Bioavailability from a Multiple Dose

A single lead candidate was selected and progressed to a multi dose-study to establish steady-state PK. Following habituation (7 days) the rats received one dose (15 mg/kg, 1 mL/kg) every 6 hours over 42 hours. Blood samples were collected (250 μl) post final dose from the tail vein over 24 hours.

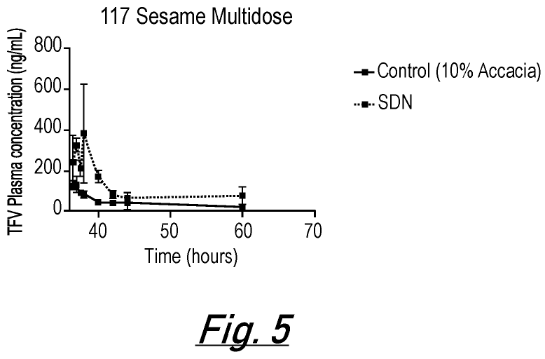

The wellbeing of each rat was monitored as in Example 6. The method of sacrifice, data collection and data analysis also matched Example 6. The results are displayed in Table 9 (standard deviations for each measurement shown in brackets) and FIG. 5 (standard deviations for each measurement shown as error bars).

TABLE 9

| | | Cₘₐₓ | Cₘᵢₙ | AUC |
|---|---|---|---|---|
| Formulation | Stabilisers | (ng/mL) | (ng/mL) | (ng/h/mL) |
| Control | 10% Acacia | 144.5 (34.36) | 18.4 (6.21) | 969.5 (367.01) |
| 117 | AOT + | 353.8 (42.17) | 74.9 (44.14) | 2311.3 (432.93) |
| Sesame | Lauroglycol FCC | | | |

At steady state the PK of 117 sesame demonstrated significantly higher plasma exposure (Cₘₐₓ 2.4 fold increase (P=0.004), Cₘᵢₙ 4.1 fold increase (P=0.024) and AUC 2.4 fold increase (P=0.026)). These data highlight the potential for dose reduction and long-acting therapeutic effects by the use of liquid compositions comprising stabilised particulates of at least one active species in an oil. In particular, they highlight the potential for dose reduction and long-acting therapeutic effects by the use of liquid compositions comprising stabilised particulates of TDF in an oil.

1. A method of producing a liquid composition comprising stabilised particulates of at least one active species in an oil, the active species being selected from nucleoside analogues and nucleotide analogues, the method comprising the steps of:
dissolving the at least one active species into a first solvent to form a first solution;
dissolving one or more stabilisers into a second solvent to form a second solution, wherein the first and second solvents are miscible and the at least one active species is insoluble in the second solvent;
combining the first and second solutions to form a suspension of stabilised particulates of the at least one active species suspended in the mixed solvent;
adding an oil to the particulate suspension to form a liquid mixture, wherein the stabilised particulates are insoluble in the oil; and
removing the first and second solvents from the liquid mixture to form the liquid composition.

2. A method according to claim 1, wherein the first solvent comprises a protic solvent and the second solvent comprises an aprotic solvent.

3. A method according to claim 2, wherein the first solvent comprises methanol and the second solvent comprises dichloromethane.

4. A method according to any preceding claim, wherein the total concentration of the at least one active species in the first solution is between about 10 and 100 mg/mL, preferably at least about 30 mg/mL, further preferably at least about 50 mg/mL, yet further preferably at least about 70 mg/mL and most preferably at least about 80 mg/mL.

5. A method according to any preceding claim, wherein the concentration of the stabilisers in the second solution is between about 1 and 30 mg/mL, preferably at least about 5 mg/mL, further preferably at least about 10 mg/mL, yet further preferably at least about 15 mg/mL and most preferably at least about 20 mg/mL.

6. A method according to any preceding claim, wherein the volume ratio of the first and second solutions on mixing is between about 2:1 to 1:10, preferably between about 1:1 and 1:6 and most preferably about 1:4.

7. A method according to any preceding claim, wherein the first and second solutions are agitated during the combining step.

8. A method according to any preceding claim, wherein the first and second solvents are removed by lyophilisation.

9. A method according to any preceding claim, wherein the nucleoside analogues are selected from adenosine analogues and guanosine analogues.

10. A method according to any of claims 1-8, wherein the nucleotide analogues are selected from adenosine monophosphate analogues and guanosine monophosphate analogues.

11. A method according to claim 10, wherein the adenosine monophosphate analogues are selected from tenofovir prodrugs.

12. A method according to claim 11, wherein the tenofovir prodrug is selected from tenofovir disoproxil, tenofovir alafenamide, their salts or combinations thereof.

13. A method according to claim 12, wherein the tenofovir disoproxil salt is tenofovir disoproxil fumerate.

14. A method according to claim 12, wherein the tenofovir alafenamide salts is tenofovir alafenamide fumurate.

15. A method according to any preceding claim, wherein the one or more stabilisers are surfactants.

16. A method according to claim 15, wherein the one or more surfactants are anionic surfactants.

17. A method according to claim 16, wherein the anionic surfactants are sulfonate salts, preferably sulfosuccinate salts.

18. A method according to claim 17, wherein the sulfosuccinate salts are selected from dioctyl sodium sulfosuccinate, dioctyl potassium sulfosuccinate, dioctyl calcium sulfosuccinate or combinations thereof.

19. A method according to any of claims 15-18, wherein further stabilisers are selected from polyoxyethylene (2) stearyl ether (Brij™ S2), propylene glycol monocaprylate (type II) (Capryol™ 90), propylene glycol monocaprylate (type I) (Capryol™ PGMC), propylene glycol dicaprylocaprate (Labrafac™ PG), apricot kernel oil PEG-6 esters (Labrafil™ M 1944 CS), corn oil PEG-6 esters (Labrafil™ M 2125 CS), caprylocaproyl polyoxyl-8 glycerides (Labrasol™), propylene glycol monolaurate (type II) (Lauroglycol™ 90), propylene glycol monolaurate (type I) (Lauroglycol™ FCC), glyceryl monolinoleate (Maisine™ 35-1), propylene glycol monopalmitostearate (Monosteol™), glycerol mono-oleate (Pecol™), polyglyceryl-3 diisostearate (Plurol™ Diisosteraque), polyglyceryl-6 dioleate (Plurol™ Olique), sorbitan oleate (Span™ 80), polyoxyethylenesorbitan monopalmitate (Tween™ 40), polyethylene glycol sorbitan monooleate (Tween™ 80) and combinations thereof.

20. A method according to any preceding claim, wherein the oil is selected from natural oils, mineral oils, synthetic oils, silicone oils and mixtures thereof.

21. A method according to claim 20, wherein the natural oil is selected from peanut oil, soy bean oil, sesame oil, safflower oil, vegetable oil, avocado oil, rice bran oil, jojoba oil, Babassu oil, palm oil, coconut oil, castor oil, cotton seed oil, olive oil, flaxseed oil, rapeseed oil and mixtures thereof.

22. A method according to any of the preceding claims, wherein the oil is biocompatible.

23. A method according to any of the preceding claims, wherein the one or more stabilisers comprise AOT and optionally a further stabiliser.

24. A method according to claim 23, wherein the further stabiliser is propylene glycol monolaurate (type I) (Lauroglycol™ FCC) or glyceryl monolinoleate (Maisine™ 35-1).

25. A method according to claim 24, wherein the further stabiliser is propylene glycol monolaurate (type I) (Lauroglycol™ FCC).

26. A method according to any of claims 23-25, wherein the oil is selected from peanut oil, sesame oil, coconut oil and soybean oil.

27. A method according to claim 26, wherein the oil is sesame oil.

28. A liquid composition comprising stabilised particulates of at least one active species in an oil obtained by the method of any one of the preceding claims.

29. A liquid composition comprising stabilised particulates of at least one active species in an oil, the active species being selected from nucleoside analogues and nucleotide analogues.

30. A liquid composition according to claim 29, wherein the nucleoside analogues are selected from adenosine analogues or guanosine analogues.

31. A liquid composition according to claim 29, wherein the nucleotide analogues are selected from adenosine monophosphate analogues or guanosine monophosphate analogues.

32. A liquid composition according to claim 31, wherein the adenosine monophosphate analogues are selected from tenofovir prodrugs.

33. A liquid composition according to claim 32, wherein the tenofovir prodrug is selected from tenofovir disoproxil, tenofovir alafenamide, their salts or combinations thereof.

34. A liquid composition according to claim 33, wherein the tenofovir disoproxil salt is tenofovir disoproxil fumerate.

35. A liquid composition according to claim 33, wherein the tenofovir alafenamide salts is tenofovir alafenamide fumurate.

36. A liquid composition according to claims 29-35, wherein the oil is selected from natural oils, mineral oils, synthetic oils, silicone oils and mixtures thereof.

37. A liquid composition according to 36, wherein the natural oil is selected from peanut oil, soy bean oil, sesame oil, safflower oil, vegetable oil, avocado oil, rice bran oil, jojoba oil, Babassu oil, palm oil, coconut oil, castor oil, cotton seed oil, olive oil, flaxseed oil, rapeseed oil and mixtures thereof.

38. A liquid composition according to claim 36 or claim 37, wherein the oil is biocompatible.

39. A liquid composition according to any of claims 28-38, wherein the nanoparticles comprise about 5-95% active species , preferably about 30-90% active species and most preferably about 60-85% active species by mass.

40. A liquid composition according to any of claims 38-39, wherein the liquid composition has a particulate concentration of at least 1 mg/mL, preferably at least 10 mg/mL, more preferably at least 40 mg/mL and most preferably at least 60 mg/mL.

41. A liquid composition according to any of claims 28-40, wherein the oil is selected from peanut oil, sesame oil, coconut oil and soybean oil.

42. A liquid composition according to claim 41, wherein the oil is sesame oil.

43. A pharmaceutical or veterinary composition in liquid dosage form comprising a liquid composition according to any one of claims 28-42, and optionally one or more additional (pharmaceutically acceptable) excipients.

44. A pharmaceutical or veterinary composition according to claim 43, wherein the pharmaceutical or veterinary composition further comprises additional pharmacologically active compounds.

45. A pharmaceutical or veterinary composition according to claim 43 or claim 44, wherein the pharmaceutical or veterinary composition is in an intramuscularly-injectable and/or subcutaneously-injectable form.

46. A pharmaceutical or veterinary composition according to any of claims 43-45, wherein the pharmaceutical or veterinary composition forms a depot at the injection site.

47. An injectable pharmaceutical or veterinary composition according to any of claims 43-46, wherein the injectable is provided in the form of a prefilled syringe.

48. A pharmaceutical or veterinary composition according to claim 43 or claim 44, wherein the pharmaceutical or veterinary composition is in a form suitable to be administered orally.

49. A pharmaceutical or veterinary composition according to claim 48, wherein the pharmaceutical or veterinary composition is in the form of a filled capsule.

50. A pharmaceutical or veterinary composition according to claim 49, wherein the pharmaceutical or veterinary composition is in the form of a gel capsule.

51. A pharmaceutical or veterinary composition according to claim 48, wherein the pharmaceutical or veterinary composition is in the form of a syrup.

52. A liquid composition according to any one of claims 28-42, or a pharmaceutical or veterinary composition according to any one of claims 43-51, for use as a medicament.

53. A liquid composition according to any one of claims 28-42, or a pharmaceutical or veterinary composition according to any one of claims 43-51, for use in the treatment and/or prevention of viral infections.

54. A liquid composition, or a pharmaceutical or veterinary composition, according to claim 53, wherein the viral infection is caused by HIV.

55. A method of treating and/or preventing an infection, the method comprising administering a therapeutically effective amount of a liquid composition according to any one of claims 28-42, or a pharmaceutical or veterinary composition according to any one of claims 43-51, to a patient suffering from or at risk of a viral infection.

56. A method of treating and/or preventing an infection according to claim 55, wherein the viral infection is caused by HIV.

57. A method of treating and/or preventing an infection according to claim 55 or claim 56, wherein after administration a therapeutically effective concentration of the active species is maintained for at least 24 hours, preferably at least 36 hours, preferably at least 48 hours, more preferably 72 hours and most preferably 96 hours.

58. A liquid composition according to any one of claims 28-42, or a pharmaceutical or veterinary composition according to any one of claims 43-51, for use in the treatment of cancers.

59. A method of treating cancer, the method comprising administering a therapeutically effective amount of a liquid composition according to any one of claims 28-42, or a pharmaceutical or veterinary composition according to any one of claims 43-51, to a patient suffering from cancer.

60. A method of treating cancer according to claim 59, wherein after administration a therapeutically effective concentration of the active species is maintained for at least 24 hours, preferably at least 36 hours, preferably at least 48 hours, more preferably 72 hours and most preferably 96 hours.
